(19) United States
(12) Patent Application Publication
(10) Pub. No.: US 2024/0013130 A1
(43) Pub. Date: Jan. 11, 2024

(54) SYSTEM AND METHOD FOR EVALUATING RISK FOR WORKERS

(71) Applicant: One Million Metrics Corp., New York, NY (US)

(72) Inventors: Aditya Bansal, Scarsdale, NY (US); Haytham Elhawary, Tuckahoe, NY (US); Trevor Pahigian, Brooklyn, NY (US); Galen Church, Queens, NY (US); Evan Roche, Brooklyn, NY (US); Mijael Alessandro Damian, Oceanside, NY (US); Sean Fronczak, Los Gatos, CA (US); Nilakshi Garg, Foster City, CA (US)

(21) Appl. No.: 18/241,488

(22) Filed: Sep. 1, 2023

Related U.S. Application Data
(63) Continuation of application No. PCT/US2023/021517, filed on May 9, 2023.
(60) Provisional application No. 63/342,442, filed on May 16, 2022.

(51) Int. Cl. G06Q 10/0639

(52) U.S. Cl. CPC G06Q 10/06398

(57) ABSTRACT

A computer-based method is provided for evaluating risk for workers. The method includes receiving, at a processor, a first signal from a first wearable device. The first signal is received from a first wearable device and is indicative of vertical acceleration detected by the first wearable device over time. The method then identifies, in the first signal, an indication of weightlessness at the first wearable device. Upon detecting the indication of weightlessness, the method proceeds to determine an amount of time associated with the indication of weightlessness and then determines a risk metric associated with the indication of weightlessness based on the amount of time associated with the indication of weightlessness.

### CROSS REFERENCE TO RELATED APPLICATIONS

This application is a continuation of International Application No. PCT/US2023/021517, filed May 9, 2023, which takes priority from Provisional Patent Application No. 63/342,442, filed on May 16, 2022, the contents of which are incorporated by reference herein.

### FIELD OF THE INVENTION

The systems and methods relate to evaluating risk for workers, and in particular, risk associated with jumping.

### BACKGROUND

In industries that require physical manipulation of objects or people, such as material handling, patient handling, manufacturing, or construction, workers often perform a variety of manual tasks such as lifting loads, moving loads from one location to another, pushing and pulling carts or trolleys, complex assembly and manipulation of components using specific motions and using vibration and impact tools. Among such tasks are jumps which generate impacts which can lead to injury.

Wearable technology has been used extensively in the consumer space to quantify, for example, the number of steps taken, distance traveled, length and quality of sleep and other metrics, but wearable technology has not been able to consistently evaluate safety metrics in the materials handling industry.

Many risks associated with material handling workers exist, including risks of impact during jumps. Such jumps may be, for example, from high platforms to lower surfaces, such as jumping off of loading docks or jumping off of or out of trucks.

There is a need for a simplified model that can give an initial indication of whether a particular activity constitutes a high risk for workers. There is a further need for a method that can identify and contextualize such high risks, such that corrective measures can be evaluated.

### SUMMARY

A computer-based method is provided for evaluating risk for workers. The method comprises receiving, at a processor, a first signal from a first wearable device. The first signal is received from a first wearable device and is indicative of vertical acceleration detected by the first wearable device over time.

The method then identifies, in the first signal, an indication of weightlessness at the first wearable device. Upon detecting the indication of weightlessness, the method proceeds to determine an amount of time associated with the indication of weightlessness and then determines a risk metric associated with the indication of weightlessness based on the amount of time associated with the indication of weightlessness.

In some embodiments, the first signal comprises an output of an accelerometer at the first wearable device. The indication of weightlessness may then be a reading of an absolute value of vertical acceleration below a threshold level.

In some such embodiments, the amount of time associated with the indication of weightlessness is an amount of time for which the accelerometer outputs a reading of vertical acceleration below the threshold level.

In some embodiments, the determining of the risk metric associated with the indication of weightlessness includes considering an indication of weightlessness only if the amount of time is greater than a threshold amount of time.

In some embodiments, the determination of the risk metric associated with the indication of weightlessness is based on the amount of time associated with the indication of weightlessness reduced by the threshold amount of time.

In some embodiments, the amount of time corresponds to a height from which free fall is experienced, and wherein the indication of weightlessness is considered only if it corresponds to a height of greater than a threshold height. Accordingly, the threshold height may be one foot and the indication of weightlessness may be considered only if longer than 0.25 seconds. Alternatively, the threshold height may be two feet and the indication of weightlessness may be considered only if longer than 0.35 seconds.

Alternatively, in some embodiments, the threshold height may be defined to correspond to a defined piece of equipment utilized by a user. In some such embodiments, the threshold height corresponds to a truck bed or a specified step of a ladder.

In some embodiments, the threshold height is based on which user of a plurality of potential users are wearing the first wearable device.

In some embodiments, the output of the accelerometer corresponds to local gravity during normal use and the indication of weightlessness corresponds to an output of the accelerometer lower than local gravity.

In some embodiments, the method includes identifying, in the first signal, a plurality of secondary indications of weightlessness, each of which has a corresponding associated amount of time. In such an embodiments, the method may further include determining a cumulative risk metric associated with the indication of weightlessness and the plurality of secondary indications of weightlessness, wherein the determining of the cumulative risk metric considers only indications of weightlessness longer than a threshold amount of time.

In some such embodiments, the cumulative risk metric is based on a cumulative amount of time associated with all of the indication of weightlessness and the plurality of secondary indications of weightlessness, and wherein, for any indication longer than threshold amount of time, the corresponding amount of time is reduced by the threshold amount of time prior to summing the amounts of time to determine the cumulative amount of time.

In some embodiments utilizing a cumulative metric, the determination of the cumulative risk metric is based only on the plurality of indications of weightlessness and the associated amounts of time.

In some embodiments utilizing a cumulative metric, the method further includes triggering an alert when the cumulative risk metric associated with the indication of weightlessness is determined to represent a high risk of injury.

In some embodiments, the method includes triggering an alert when the risk metric associated with the indication of weightlessness is determined to represent a high risk of injury.

In some embodiments, the determination of the risk is based only on the indication of weightlessness and the associated amount of time.

In some embodiments, the first wearable device is not calibrated after application to the user.

In some embodiments, the method includes identifying a contextual characteristic associated with the worker either simultaneous with or immediately preceding or following the indication of weightlessness.

In some such embodiments, the contextual characteristic is identified in the first signal. In some such embodiments, the contextual characteristic is a horizontal acceleration or deceleration identified prior to the indication of weightlessness. In some such embodiments, the method determines that the worker was decelerating in a vehicle prior to the indication of weightlessness based on the horizontal acceleration or deceleration identified.

In some such embodiments, an alert to a supervisor or a recommendation that a characteristic of the vehicle be modified is generated based on the contextual characteristic and the indication of weightlessness.

In some embodiments in which the contextual characteristic is identified in the first signal, the contextual characteristic is a change of gait or a failure to perform an expected movement, and wherein the contextual characteristic indicates worker injury.

In some embodiments in which a contextual characteristic is identified, the method includes receiving, at the processor, a second signal indicative of a physical characteristic of the first wearable device simultaneous with or immediately preceding or following the indication of weightlessness. The contextual characteristic is then identified in the second signal. In some such embodiments, the second signal indicates location data so as to identify a location of the indication of weightlessness.

In some such embodiments, the method includes retrieving risk metrics and associated contextual characteristics from a plurality of workers, and identifying in the contextual characteristics at least one location consistently associated with high risk.

In some embodiments, in which a contextual characteristic is identified, a physical activity is associated with the indication of weightlessness based on the contextual characteristic.

### BRIEF DESCRIPTION OF THE DRAWINGS

FIG. 1 illustrates a physical environment for implementing a method for monitoring safety and evaluating risk;

FIG. 2 is a schematic for a sensor and sensor packaging for use in implementing the method;

FIG. 3 is a wearable device for evaluating risk to a worker;

FIG. 4 illustrates a specific physical environment in which a method for evaluating risk may be implemented;

FIG. 5 illustrates the path a wearable device takes when a worker wearing the device jumps off of a raised platform such as that shown in FIG. 4;

FIGS. 6 and 7 illustrate the relationship between time and displacement when in freefall; and

FIG. 8 is a flowchart illustrating a method in accordance with this disclosure.

FIG. 9 is a flowchart illustrating a method in accordance with this disclosure.

### DETAILED DESCRIPTION OF THE PREFERRED EMBODIMENTS

The description of illustrative embodiments according to principles of the present invention is intended to be read in connection with the accompanying drawings, which are to be considered part of the entire written description. In the description of embodiments of the invention disclosed herein, any reference to direction or orientation is merely intended for convenience of description and is not intended in any way to limit the scope of the present invention. Relative terms such as “lower,” “upper,” “horizontal,” “vertical,” “above,” “below,” “up,” “down,” “top” and “bottom” as well as derivative thereof (e.g., “horizontally,” “downwardly,” “upwardly,” etc.) should be construed to refer to the orientation as then described or as shown in the drawing under discussion. These relative terms are for convenience of description only and do not require that the apparatus be constructed or operated in a particular orientation unless explicitly indicated as such. Terms such as “attached,” “affixed,” “connected,” “coupled,” “interconnected,” and similar refer to a relationship wherein structures are secured or attached to one another either directly or indirectly through intervening structures, as well as both movable or rigid attachments or relationships, unless expressly described otherwise. Moreover, the features and benefits of the invention are illustrated by reference to the exemplified embodiments. Accordingly, the invention expressly should not be limited to such exemplary embodiments illustrating some possible non-limiting combination of features that may exist alone or in other combinations of features; the scope of the invention being defined by the claims appended hereto.

This disclosure describes the best mode or modes of practicing the invention as presently contemplated. This description is not intended to be understood in a limiting sense, but provides an example of the invention presented solely for illustrative purposes by reference to the accompanying drawings to advise one of ordinary skill in the art of the advantages and construction of the invention. In the various views of the drawings, like reference characters designate like or similar parts.

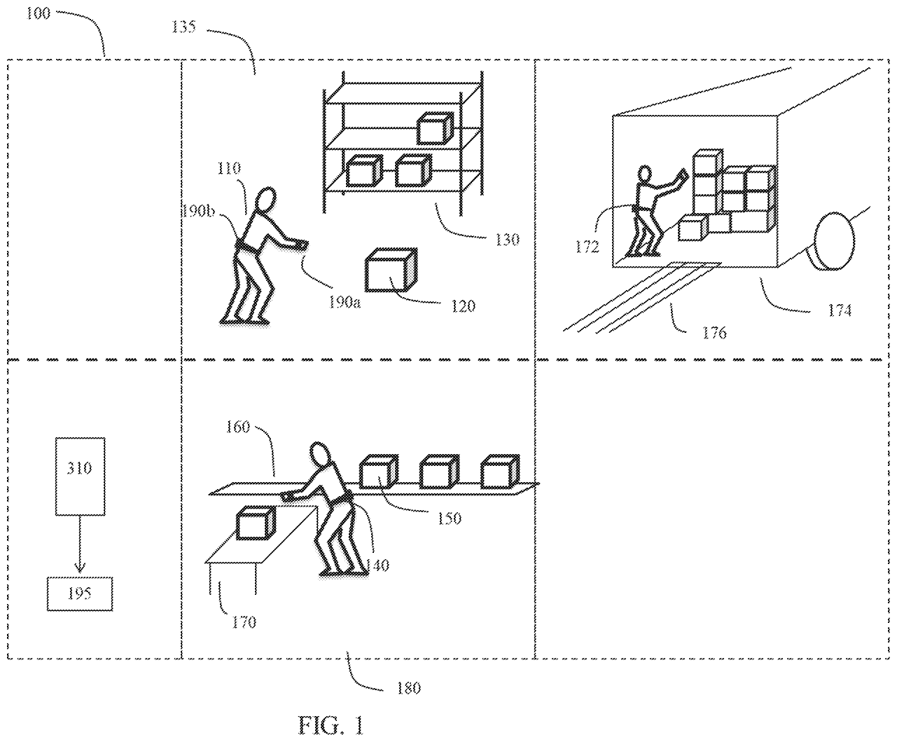

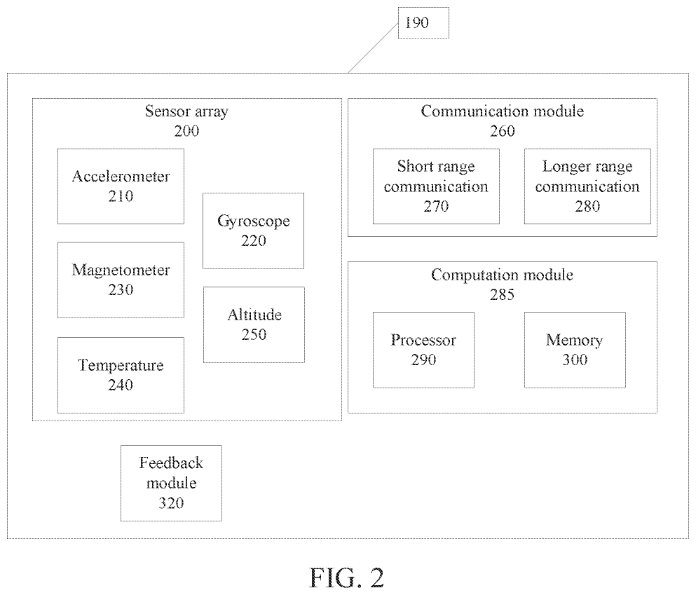

FIG. 1 illustrates a typical environment in which the system and method monitoring safety and productivity is deployed, FIG. 2 is a schematic for a sensor implementation for use in the method, and FIG. 3 is a wearable device for evaluating risk to a worker.

As shown in FIG. 1, workers, or other users of the systems and methods described herein, may be deployed to various locations within a warehouse 100 and may be required to perform a variety of material handling tasks at each location. For example, a first worker 110 may lift an object 120 from the floor to a shelf 130 in a first sector 135 within a warehouse 100, while a second worker 140 may lift a separate object 150 off of a shelf 160, rotate, and transfer it to a table 170 in a second sector 180 of the warehouse 100. Additional activities may include a worker 172 unloading a truck 174 which may include walking up and down a ramp 176, jumping, or operating machinery, among other activities. It will be understood throughout this disclosure that references to a worker are references to users wearing the wearable devices 190 discussed herein. It will be understood that in this disclosure, wearable devices and sensor devices 190, 190a, and 190b, may refer to the same devices.

Each of the workers 110, 140, 172 would typically be wearing at least one sensor device 190, and in some embodiments, two sensor devices, 190a, b for recording movement. Typically, where two sensors are provided, the sensors used may be a wrist sensor device 190a, ideally located on the wrist or forearm of the dominant hand, and a back sensor device 190b, ideally located approximately at the height of the L1 and L2 vertebrae, but other sensor device types may be implemented as well. The wrist sensor may be incorporated into a wrist device, such as a bracelet or a wristwatch, and the back device may be incorporated into a chest strap, weight belt or back brace, for example. Where only one sensor device 190 is provided, it is typically applied to a worker 110, 140, 172 on or near the worker's hip. However, the device 190 may be applied elsewhere and the necessary dimensions and measurements may be extrapolated from data recorded from the sensor device 190. The sensor device 190 may take a variety of forms, and is referred to herein as any of a sensor, a device, or a sensor device.

Accordingly, a single sensor device 190, referred to herein as a wearable device, may be used to record movement. Such a sensor may be mounted on a user's belt and may be used to predict or estimate motion of the user's back and spine based on movements of the user's hip. A system implementing such a wearable device may be trained using a machine learning predictive model trained by collecting data from sensors attached to a user's spine and comparing that data to data collected at the user's hip. After training such a predictive model, the single hip mounted wearable device 190 may be used to evaluate movement of a worker's spine.

Alternatively, in some embodiments, a simplified model may be implemented using only a single signal from a single sensor. Such a simplified model may have the benefit of returning results quickly, or in real time, requiring less processing power, and having fewer opportunities for false positives.

Accordingly, in embodiments using a single wearable device 190, the wearable device is mounted at a worker's hip, and the measurements calculated include measurements of a user's back inferred from movement of the user's hip detected by the wearable device. Such movement of the user's hip may be detected by the accelerometer 210, gyroscope 220, and altimeter 240, discussed above. In other embodiments, such measurements may be calculated based solely on a signal output from an accelerometer 210.

In some embodiments, a single primary wearable device 190 may be used and it may communicate with various sensors or transmitters on different parts of the user's body, in an environment in which the user is working, or on equipment the user is using. For example, a user may have a primary wearable device 190 that interacts with safety equipment worn by a user or with a humidity, temperature, or gas sensor located in a factory.

A server 310 may further be included in the warehouse 100 for receiving data from the wrist sensor 190a and the back sensor 190b, or the single wearable device 190, depending on the implementation, and storing records of activity performed by workers 110, 140, 172. In some embodiments, signals generated and transmitted by the wearable device 190 are received and processed by the server 310. In some embodiments, results of the methods discussed below are generated and retained by the wearable devices 190 and are used to provide immediate feedback to workers 110, 140, 172. In some embodiments, the results are transmitted to additional terminal devices 195 to be accessed by a third party, such as a manager, or by the workers themselves 110, 140, 172. While the warehouse 100 shown includes a physical server 310, it will be understood that the server may be a cloud server or may be coupled to a cloud server to maintain a platform implementing the method described.

As shown in FIG. 2, each wearable device 190 may include a sensor array 200 including a 3-axis accelerometer 210, a 3-axis gyroscope 220, a 3-axis magnetometer 230, a temperature sensor 240, and an altitude sensor 250, such as a barometric pressure sensor. Each sensor device 190 may further include a communication module 260 which may include multiple communication interfaces. For example, each sensor device 190 may have a short range communication interface 270 for enabling communications between a first sensor device 190a and a second sensor device 190b worn by a single user. The short range communication interface 270 may further be used to receive signals from additional sensors or devices on the user's body, such as safety equipment, or from sensors or other transmitters in the user's immediate environment. The wearable device 190 may further contain a longer range communication interface 280 for connecting, for example, to a Wi-Fi or cellular network. Each wearable device 190 may further include a computation module 285, including a processor 290 and a memory 300.

Accordingly, each of the sensor devices 190a, b, may communicate with each other (in embodiments where users wear multiple sensor devices), or other local devices or sensors, using the short range communication interface 270 and with the server 310 or a cloud network using the longer range communication interface 280. Signals generated by the sensor devices 190 may be processed at the individual devices, may be combined with other data acquired through the short range communication interface 270, or may be transmitted to the server 310 or another centralized platform for analysis.

The wearable device 190 may further incorporate a feedback module 320 for providing feedback to the user. For example, the feedback module 320 may include a motor for generating vibration and providing haptic feedback, audible feedback in response to the output of the method, and/or a display for visual feedback that can show immediate as well as cumulative risk exposure. Further, different levels or patterns of vibration in the context of haptic feedback may be used to indicate different alerts to the user of the device. The wearable devices 190 may further incorporate user input means by which users can control the wearable device 190. For example, the device may include modules for detecting and interpreting voice or gesture-based commands.

The wearable device 190 may have an additional module for determining location by, for example, incorporating a GPS unit or other geolocation components and processes. Alternatively, or in addition to geolocation components, the wearable device 190 may include a module for triangulating the location of workers based on proximity to known landmarks, such as beacons.

The sensor devices 190 may further include batteries for providing power to the various modules therein. The sensor devices may further incorporate LEDs, displays, or other methods for delivering feedback to the workers 110, 140, 172 wearing the sensors. For example, the device may utilize a display to display the risk metrics, or a goal, rank or other relevant information like battery and signal status. The display may be touch sensitive in order to provide a user interface by way of the display. Other information displayed can be error or warning messages when a worker is detected to not be wearing the device correctly, or in a variety of other scenarios discussed below in more detail. The device can also show information like number of steps taken by a worker, calories burned, active hours in the shift, current time and the time to next break etc. This information can be shown when a worker requests it, at regular intervals, or automatically when one of the methods described below are used to identify a relevant or hazardous situation.

In some embodiments, the user interface is replaced by, or supplemented by, a separate portable device or an application for use on a smartphone. In such a case, when an alert is triggered, such alert may be transmitted to a user on his smartphone.

In some embodiments, the wearable device 190 is used to implement a high risk jump model that determines situations where there can be a significant risk to human body joints, like ankles, knees, and pelvis. The specific jumps that have been considered high risk are the ones that primarily happen in industrial workplaces like manufacturing, warehouses etc., and also inclusive of the ones that happen while performing delivery functions on road by parcel delivery and last mile delivery drivers. These jumps can be also be viewed as from a higher platform to ground or a lower platform.

Based on a study by Washington State Department of Labor and Industries, a person jumping or falling from a height of 4 feet would hit the ground with a force of between 7 and 12 times their body weight. For example, a 200 lb. person would hit the ground with a force of 1,400 to 2,400 Lbs.

Note that the impact to someone's ankles, knees, back etc. is proportional not only to their body weight but also the jumping height. One can also note that the actual impact to body parts depends on several factors like the health history of the person, the kind of shoes they were wearing, landing form, flatness of the landing area etc. Hence, the impact, and associated risk, can vary between person to person, and also each jump can be different. However, the biggest contributor to impact is the height from which someone jumped.

The wearable device 190 is therefore provided with, at least, an accelerometer 210, which is then used to generate a proxy value allowing a model to estimate the height H of a raised platform.

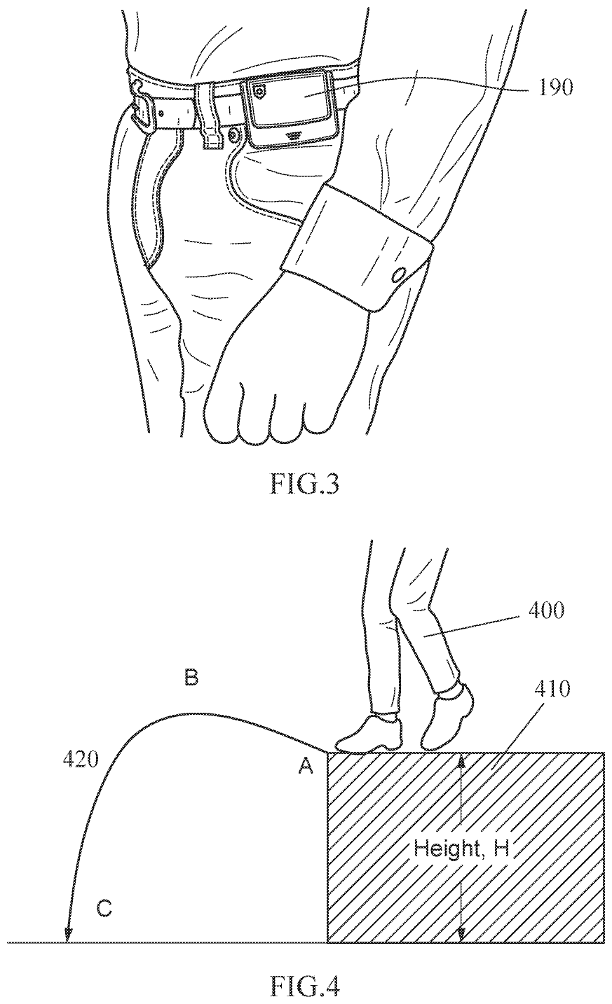

FIG. 4 illustrates a specific physical environment in which a method for evaluating risk may be implemented. As shown, a user 400 of the system may stand on a raised platform 410. In the context of the environment of FIG. 1, the user 400 may be a worker 172 unloading a truck 174, and the truck bed or trailer may be the raised platform 410. Similarly, the raised platform 410 may be a ladder, a loading dock, or some other raised platform in a workplace. The user 400 may then be at a height H on top of the platform 410.

In such a scenario, the user 400 may choose to leave the platform 410 by jumping. The path 420 followed by the user's 400 feet may then include points A, B, and C.

FIG. 5 illustrates the path a wearable device takes when a worker 400 wearing the device 190 jumps off of a raised platform 410 such as that shown in FIG. 4. The device 190 typically includes an accelerometer 210, such as a 3-axis accelerometer attached to a user's 400 belt to capture the motion of their hips or pelvis as they jump down from the higher platform 410.

As shown in FIG. 5, the device 190 follows a path 500 similar to the curve A-B-C representing the path 420 followed by the user's feet. A vertical acceleration detected by the accelerometer 210 of the device 190 is computed and is shown as the signal ay. This signal captures vertical acceleration during the jump.

It is noted that the signal av need not be aligned with any single axis X, Y, Z of the accelerometer 210 of the device 190. As such, the signal is agnostic as to how the device 190 is placed on the user 400 as far as it captures the motion of the user's body during the jump. Similarly, the signal is agnostic as to the orientation of the accelerometer 210. As such, the signal measured by the accelerometer 210 may be positive or negative, and as noted below, determinations related to the signal may be based on an absolute value of the signal and the sign of any value in the signal may be ignored.

When a person stands on the higher platform 410, i.e., at point A of the path 420, the vertical acceleration av is generally equal to approximately 1 g, where g is the gravitational acceleration. On earth, g is approximately equal to 9.8 m/s\hat{ }2. This is the acceleration measured by the accelerometer 210 attached to the user 410 when not moving.

When the user 400 jumps and is between points B and C on path 420, the person's body experiences free fall for a short duration. During this time, the person and the device 190 and corresponding accelerometer 210 are all in free fall, and the vertical acceleration registered by the accelerometer is approximately 0 g. In other words, no acceleration is measured at the device 190.

When the user 400 lands on the ground at point C, free fall ends, and the accelerometer 210 again experiences vertical acceleration as approximately 1 g. This means that vertical acceleration av goes from 1 g at point A to 0 g during free fall and finally returns to 1 g on landing at point C.

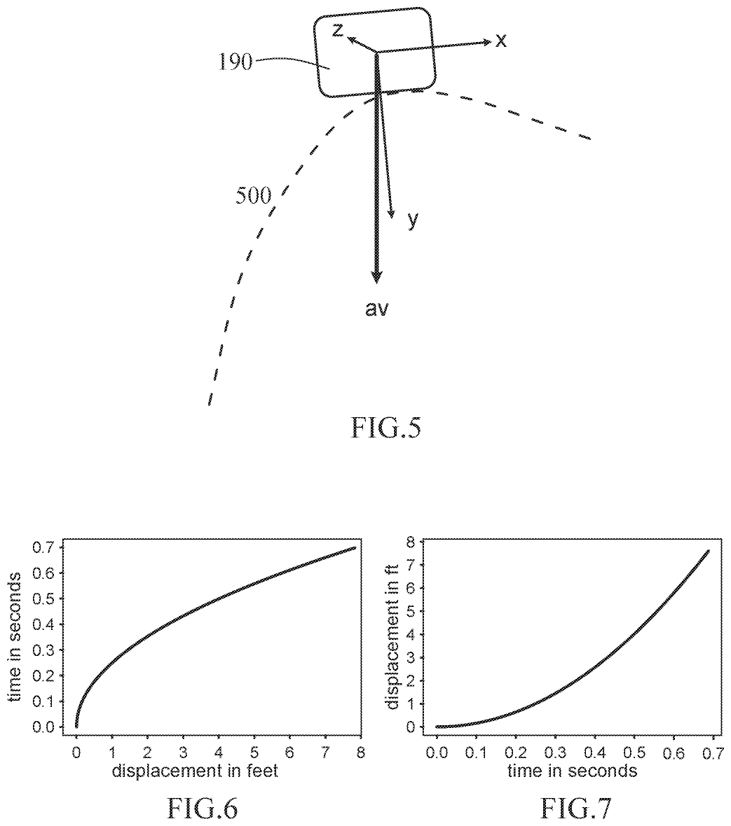

FIGS. 6 and 7 illustrate the relationship between time and displacement when in freefall. As shown, the duration for which vertical acceleration is 0 g is proportional to the height from which the user 400 has experienced free fall. Accordingly, the amount of time in free fall can be correlated with the height H of a platform 410 from which the user 400 has jumped. The higher the platform, the longer the user 400 would be in free fall.

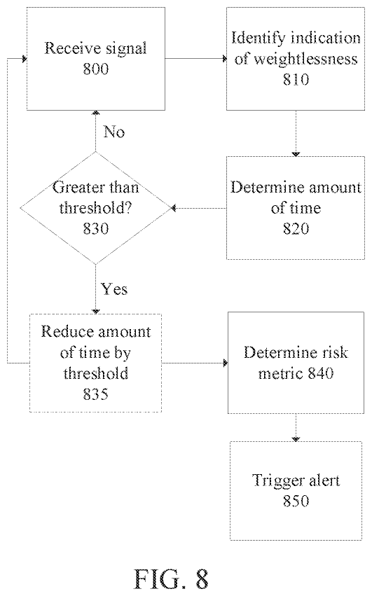

FIG. 8 is a flowchart illustrating a method in accordance with this disclosure. As shown, and as discussed above, the method is a computer-based method for evaluating risk for workers. The particular embodiment provided in FIG. 8 relates to evaluating risk associated with a worker jumping. Workers may be provided with wearable devices 190, such as those discussed above, and such devices may include accelerometers 210 for determining acceleration associated with the device at any given time.

The method therefore includes receiving (800), at a processor 290, a first signal from a first wearable device 190 indicative of vertical acceleration detected by the first device over time. The first signal may be, for example, the output of an accelerometer 210 of the first wearable device 190. It is understood that while the method is described herein as receiving the first signal and processing it at the processor 290 of the first wearable device 190, the method may similarly process the data elsewhere. For example, the first signal may be received (at 800) at the server and processed there 310.

The method then identifies (810), in the first signal, an indication of weightlessness of the first wearable device 190. This may be a reading of an absolute value of vertical acceleration detected at the accelerometer 210 below a threshold level. It is noted that true free fall, corresponding to 0 g, would not typically be recorded, as accelerometers are generally noisy. Accordingly, in the scenario illustrated in FIG. 4, the accelerometer 210 would not respond to free fall by switching quickly from 1 g at point A to 0 g at point B and back to 1 g on landing at point C. Instead, a threshold value is set, and any recorded value having an absolute value below that threshold value is therefore determined to represent free fall.

In the embodiment shown, the specific orientation of the wearable device 190 is not considered, and as such, the wearable device is considered to be in free fall, and therefore weightless, if all readings recorded at the accelerometer 210 are below the threshold level. Because orientation may determine the sign of the signal, vertical acceleration may be positive or negative depending on the orientation of the wearable device 190 or the orientation of the accelerometer 210 relative to the wearable device. As such, the method typically ignores the sign and relies on the absolute value of the vertical acceleration. As such, in this disclosure, it will be understood that when referencing a value determined to be below a threshold, such a value may be an absolute value.

In some embodiments, the method may monitor a value representing vertical acceleration, and may identify an indication of weightlessness when such a value crosses a threshold.

Upon detecting the indication of weightlessness (at 810), the method proceeds with determining an amount of time associated with the indication of weightlessness (820). The amount of time determined (at 820) may be an amount of time for which the accelerometer 210 outputs a reading of vertical acceleration below the threshold level.

In some embodiments, the method only considers an indication of weightlessness to represent a risk if the amount of time is greater than a threshold amount of time. As such, the method may determine if the amount of time associated (at 820) with the indication of weightlessness is greater than the threshold amount of time (830).

A risk metric associated with the indication of weightlessness is then determined (840) based on the amount of time associated with the indication of weightlessness. As noted above, in some embodiments, the risk metric may only be determined (at 840), if the time associated with the indication of weightlessness was determined to be greater than the threshold (at 830). If the amount of time is not greater than the threshold, the method continues to receive values (at 800) and continues to identify additional indications of weightlessness.

As such, the risk metric may be presented as:

risk=f(duration of free fall i.e., av<threshold)

Accordingly, the methods can be used to determine risks associated with jumps, but only jumps likely to generate risk are considered. During normal daily activities, many motions, such as jogging, or jumping from the last steps of a staircase may cause a user's 400 body to briefly experience free fall. However, the height in these motions is quite small and can be considered generally safe, depending on several other human factors. For example, OSHA recommends that stair height and ladder spacing should be less than 1 ft. Accordingly, the methods described herein may only consider heights larger than those approved generally by OSHA.

Accordingly, and as shown in FIGS. 6 and 7, the amount of time associated (at 820) with the indication of weightlessness corresponds to a height from which free fall is experienced. The indication of weightlessness may only be considered if it corresponds to a height greater than a threshold height. Such a threshold height may be one foot, to correspond to the OSHA recommendation, and as such an indication of weightlessness may only be considered if longer than 0.2 seconds. More precision is possible, and such a threshold may therefore be 0.25 seconds. Similarly, the model may allow for longer periods of free fall. The threshold height may, for example be two feet and the indication of weightlessness may then only be considered if longer than 0.35 seconds.

In some embodiments, a user 400 may be approved to work with a defined piece of equipment. For example, a user may be approved to work with a particular loading dock, truck bed, or ladder, and may be permitted to jump down from that height, but no more. Similarly, the user may be permitted to jump from the second step of a ladder but no higher step. As such, the threshold height may be set to correspond to a particular piece of equipment utilized by the user 400.

Further, in some embodiments, certain users 400 may be qualified to jump from different heights due to health or build or due to confirmation that they are able to jump with proper form. As such, the threshold height may be based on which user 400 of a plurality of potential users are wearing the first wearable device 190. For example, a user 400 who has taken training on proper jumping technique may receive approval to jump from a higher threshold height for some period of time following the training.

In some embodiments, where the amount of time associated (at 820) with the indication of weightlessness is considered in determining the risk metric, the determination may be based on the amount of time associated with the indication of weightlessness reduced by the threshold amount of time (at 835).

In some embodiments, the risk metric (determined at 840) may be determined based only on the indication of weightlessness and the associated amount of time. In some embodiments, particularly when the input to the determination of the risk metric is limited, no calibration of the first wearable device 190 is necessary after application of the device to the user 400.

In some embodiments, following a determination that a particular jump should be considered in the determination of the risk metric (at 840), such as by confirming that the length of time associated with an indication of weightlessness was greater than the threshold amount of time (at 830), the method continues to receive and monitor the first signal (at 800) and identifies (at 810) additional secondary indications of weightlessness. Such monitoring may continue while the method continuously determines a current risk metric (at 840) associated with already detected indications of weightlessness.

In such embodiments, the method may monitor a user 400 over an extended period of time, and therefore may evaluate metrics associated with multiple jumps performed over the course of a shift, day, or other period of time. Such indications may therefore be identical in principle to the first indication, as each such indication simply represents a jump, and therefore may each have a corresponding associated amount of time (determined at 820).

The risk metric (calculated at 840) may then be a cumulative risk metric determined based on each of the indication of weightlessness and the plurality of secondary indications of weightlessness. As discussed above, each indication of weightlessness may be considered independently to determine if the associated length of time is greater than the threshold (at 830), and as such, the cumulative risk metric may consider only indications of weightlessness longer than the threshold amount of time.

In some embodiments, the cumulative risk metric is based on a cumulative amount of time associated with all of the indications of weightlessness and the plurality of secondary indications of weightlessness taken together. In such embodiments, as noted above, the amount of time considered for each indication of weightlessness for the purpose of calculating the risk metric (at 840) may be reduced by the threshold amount of time prior to summing the amounts of time to determine the cumulative amount of time.

In some embodiments, the determination of the cumulative risk metric is based only on the plurality of indications of weightlessness and the associated amounts of time.

In some embodiments, upon determining from the risk metric or cumulative risk metric (calculated at 840) that an indication of weightlessness or multiple indications of weightlessness represent a high risk of injury, the method may trigger an alert (850) to the user 400 or to a supervisor indicating such risk. Such an alert may be issued on the wearable device 190 itself, or at a linked device, such as a user's smartphone, or at a server 310 monitored elsewhere.

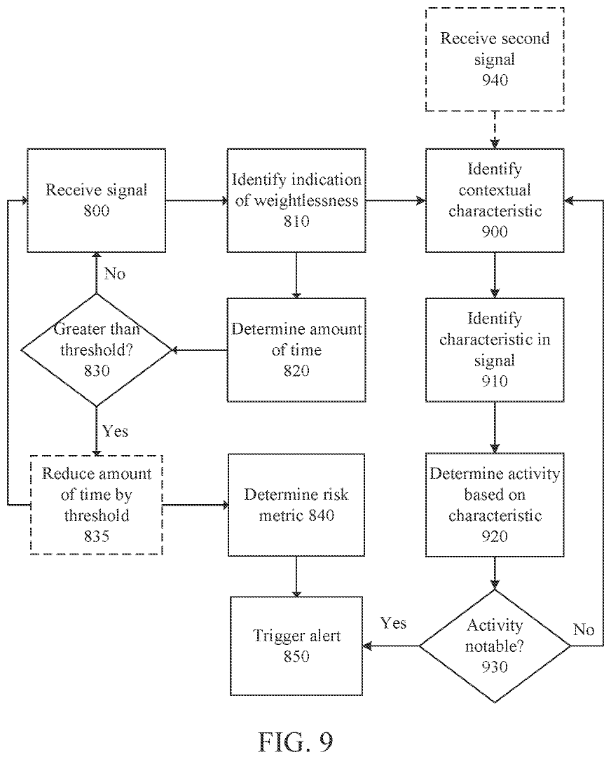

FIG. 9 is a flowchart illustrating a method in accordance with this disclosure. Generally, the method of FIG. 9 is similar to that presented in FIG. 8, and similar steps shown proceed generally as described above with respect to FIG. 8.

In some embodiments, upon identifying an indication of weightlessness (at 810), the method may extend the jump model described in order to include context. In order to do so, the method may further comprise identifying a contextual characteristic (at 900) associated with the worker 400. The contextual characteristic may be simultaneous with or immediately preceding or following the indication of weightlessness identified (at 810).

Typically, the contextual characteristic would be identified (at 900) following the identification of an indication of weightlessness (at 810), and once identified, the contextual characteristic would provide some context for a moment of time either simultaneous with or immediately before or after the corresponding indication of weightlessness. As such, the context provided is associated with a particular indication of weightlessness. Accordingly, when a specific jump is identified, context may be provided for association with that particular jump.

In some embodiments, the contextual characteristic (900) is identified in the first signal (received at 800). For example, the first signal may be accelerometer data in multiple dimensions, and the contextual characteristic may be a horizontal acceleration or deceleration identified (910) in the accelerometer data. Where the contextual characteristic is identified in the first signal at a time immediately prior to the indication of weightlessness (at 810), such acceleration or deceleration (at 910) may indicate acceleration or deceleration of the worker 400 immediately prior to a jump identified by the indication of weightlessness.

For example, a deceleration identified (at 910) may indicate that a worker was decelerating in a vehicle prior to the indication of weightlessness. Accordingly, the method may proceed to determine (920) that the worker was decelerating in such a vehicle prior to the indication of weightlessness based on horizontal deceleration identified.

Similarly, the method may determine that the worker was accelerating to jump over an obstacle or the like prior to the indication of weightlessness based on horizontal acceleration identified.

Once a contextual characteristic has been identified (at 900) and a corresponding activity has been determined (at 920), the method may proceed to determine if the activity identified is notable (at 930), and if so, trigger an alert (at 850) to the user 400 or to a supervisor indicating the identification of an activity collated with the indication of weightlessness identified (at 810). If context is determined not to be notable (at 930), then the method may proceed to monitor context associated with additional indications of weightlessness.

The alert triggered (at 850) may include or comprise a recommendation associated with the activity identified. Accordingly, if the activity identified (at 920) is the deceleration of a vehicle immediately prior to the indication of weightlessness, the method may determine a step out of a vehicle is too tall, and that workers are therefore required to step down too far. Similarly, if the activity is identified (at 920) as deceleration with some more time between the activity and the indication of weightlessness, the method may determine that the truck bed is too tall, rather than steps out of the cabin. Accordingly, the alert (at 850) may include a recommendation that a characteristic of the vehicle be modified. This may be to introduce steps, or additional or improved steps and/or handrails, so as to avoid workers jumping and help them step down safely.

In some embodiments, the identified characteristic in the signal (at 910) may be an activity identified following the indication of weightlessness (at 810). For example, the activity determined (at 920) may be a limp or other indication of injury following a jump. Accordingly, the activity may be a failure of a worker to perform an expected movement, and may in turn indicate a worker injury.

In some embodiments, the method described may include receiving, at the processor 290, a second signal (at 940) independent of the first signal received (at 800). The second signal may be indicative of a physical characteristic of the first wearable device 190 simultaneous with or immediately preceding or following the indication of weightlessness. In such an embodiment, the contextual characteristic is then identified (at 900) in the second signal and collated with the indication of weightlessness identified (at 810) in the first signal.

In some embodiments, the second signal (received at 940) indicates location data, so as to identify a location of the indication of weightlessness. Accordingly, the characteristic identified (at 910) may be a location at which the jump defined by the indication of weightlessness occurred. The location data may be drawn, for example, from GPS data or from beacons arranged within a warehouse or may be otherwise derived from context.

In this manner, location context may be added to each indication of weightlessness (identified at 810). Accordingly, the method may proceed to identify a location on a worker's route at which high risk jumps occurred or locations within a warehouse at which workers tend to take risks.

In some embodiments, the contextual characteristic identified (at 900) may simply be a time of day or a time within a worker shift at which the indication of weightlessness (identified at 810) occurred). Accordingly, if a worker takes higher risk jumps later in a shift or later in a day, such jumps may be attributed to fatigue or lack of concentration, and the method may generate recommendations to shorten shifts, increase alerts to workers later in shifts, or reschedule or reconfigure shifts to avoid locations where steps or ladders are used later in shifts.

In some embodiments, the contextual characteristics are only identified (at 900) or determined to be notable (at 930) where a determined risk metric 840 is above a threshold level. Accordingly, context may only be recorded or an alert may only be generated where a jump is first determined to be high risk.

In some embodiments, the method may retrieve risk metrics (840), or indications of weightlessness (810), and associated contextual characteristics (900) from multiple workers. The method may then identify in the contextual characteristics at least one location consistently associated with a high risk. Accordingly, the system may identify a warehouse location at which some physical issue must be resolved, such as a ladder to be repaired or reconfigured, or a route location at which trucks consistently encounter an issue, such as an incorrectly configured loading dock.

In some embodiments, a specific activity may be associated with the indication of weightlessness (at 810) based on the contextual characteristic (identified at 900). For example, as noted above, where a jump is preceded by deceleration, the indication of weightlessness may be identified as stepping out of a truck.

While the second signal discussed herein is discussed in terms of location data, it is noted that different types of data may be provided in the second signal in addition to or in place of the location data. Accordingly, the method may be tailored to specific scenarios that a user of the method described may wish to monitor.

Accordingly, the method may be used in various scenarios in order to determine if workers are safely descending or jumping off of a raised surface, and to provide context to supervisors.

For example, in the context of parcel delivery, the rate of jumping off of a truck may be associated with the time of the shift. The chances of a worker jumping may be higher when they are at the end of their shift and trying to quickly finish a route. Accordingly, the method may be provided with route information, and the contextual characteristic may be an element of a worker's route information. The method may then be used to determine if a route has some hazards or environmental factor that forces workers to jump instead of descending safely.

The method may then generate alerts and/or recommendations as to how to increase safety of routes, such as rearranging routes so as to locate more hazardous locations earlier in a shift or to modify the physical parameters of a specific route location. Similarly, the method may be used to assign safety equipment, or an additional ladder, to workers traveling a specific route with a known hazard.

Similarly, in the context of warehouses, workers may need to use ladders or tall forklifts to do their work. Detecting high risk jumps in warehouses may be associated with workers jumping off forklifts or skipping the last few steps of a ladder. As noted above, the contextual characteristic may be utilized to determine if a worker had previously been riding in a forklift or is in a location associated with a ladder.

Similarly, in the context of construction projects, workers may be regulated in terms of wearing harnesses while on raised surfaces. However, even at a single level, turf may be irregular and, for example, girders or concrete blocks may be lying in walking paths. As a result, workers may be required to jump over some debris, and the method may identify consistent issues and thereby recommend moving debris.

In some embodiments, following implementation of the method, any context identified (at 900) and associated with high risk instances of indications of weightlessness (at 810) may be monitored, so as to determine if specific context is a recurring issue. For example, specific locations on delivery routes may be monitored for high risk. Similarly, specific warehouse locations or times of day or shift may be monitored. In some embodiments, if recommendations were generated (at 850) and changes were implemented, the method may continue to monitor similar context (at 900) in order to determine if the changes implemented were effective or to determine if further changes are warranted.

In the embodiments discussed, the contextual characteristics may be identified (at 900) in real time, or, in some embodiments, contextual context may be added later during review of earlier results of the method. Accordingly, in some embodiments, context is only sought or evaluated once an indication of weightlessness (at 810) has already been determined to generate a high risk metric (at 840). Alternatively, context may be monitored in real time.

Embodiments of the subject matter and the operations described in this specification can be implemented in digital electronic circuitry, or in computer software, firmware, or hardware, including the structures disclosed in this specification and their structural equivalents, or in combinations of one or more of them. Embodiments of the subject matter described in this specification can be implemented as one or more computer programs, i.e., one or more modules of computer program instructions, encoded on computer storage medium for execution by, or to control the operation of, data processing apparatus. Alternatively or in addition, the program instructions can be encoded on an artificially generated propagated signal, e.g., a machine-generated electrical, optical, or electromagnetic signal that is generated to encode information for transmission to suitable receiver apparatus for execution by a data processing apparatus. A computer storage medium can be, or be included in, a computer-readable storage device, a computer-readable storage substrate, a random or serial access memory array or device, or a combination of one or more of them. Moreover, while a computer storage medium is not a propagated signal, a computer storage medium can be a source or destination of computer program instructions encoded in an artificially generated propagated signal. The computer storage medium can also be, or be included in, one or more separate physical components or media (e.g., multiple CDs, disks, or other storage devices).

The operations described in this specification can be implemented as operations performed by a data processing apparatus on data stored on one or more computer-readable storage devices or received from other sources.

The term “data processing apparatus” and like terms encompass all kinds of apparatus, devices, and machines for processing data, including by way of example a programmable processor, a computer, a system on a chip, or multiple ones, or combinations, of the foregoing. The apparatus can include special purpose logic circuitry, e.g., an FPGA (field programmable gate array) or an ASIC (application specific integrated circuit). The apparatus can also include, in addition to hardware, code that creates an execution environment for the computer program in question, e.g., code that constitutes processor firmware, a protocol stack, a database management system, an operating system, a cross-platform runtime environment, a virtual machine, or a combination of one or more of them. The apparatus and execution environment can realize various different computing model infrastructures, such as web services, distributed computing and grid computing infrastructures.

A computer program (also known as a program, software, software application, script, or code) can be written in any form of programming language, including compiled or interpreted languages, declarative or procedural languages, and it can be deployed in any form, including as a standalone program or as a module, component, subroutine, object, or other unit suitable for use in a computing environment. A computer program may, but need not, correspond to a file in a file system. A program can be stored in a portion of a file that holds other programs or data (e.g., one or more scripts stored in a markup language document), in a single file dedicated to the program in question, or in multiple coordinated files (e.g., files that store one or more modules, sub programs, or portions of code). A computer program can be deployed to be executed on one computer or on multiple computers that are located at one site or distributed across multiple sites and interconnected by a communication network.

The processes and logic flows described in this specification can be performed by one or more programmable processors executing one or more computer programs to perform actions by operating on input data and generating output. The processes and logic flows can also be performed by, and apparatus can also be implemented as, special purpose logic circuitry, e.g., an FPGA (field programmable gate array) or an ASIC (application specific integrated circuit).

Processors suitable for the execution of a computer program include, by way of example, both general and special purpose microprocessors, and any one or more processors of any kind of digital computer. Generally, a processor will receive instructions and data from a read only memory or a random access memory or both. The essential elements of a computer are a processor for performing actions in accordance with instructions and one or more memory devices for storing instructions and data. Generally, a computer will also include, or be operatively coupled to receive data from or transfer data to, or both, one or more mass storage devices for storing data, e.g., magnetic, magneto optical disks, or optical disks. However, a computer need not have such devices. Moreover, a computer can be embedded in another device, e.g., a mobile telephone, a personal digital assistant (PDA), a mobile audio or video player, a game console, a Global Positioning System (GPS) receiver, or a portable storage device (e.g., a universal serial bus (USB) flash drive), to name just a few. Devices suitable for storing computer program instructions and data include all forms of non volatile memory, media and memory devices, including by way of example semiconductor memory devices, e.g., EPROM, EEPROM, and flash memory devices; magnetic disks, e.g., internal hard disks or removable disks; magneto optical disks; and CD ROM and DVD-ROM disks. The processor and the memory can be supplemented by, or incorporated in, special purpose logic circuitry.

To provide for interaction with a user, embodiments of the subject matter described in this specification can be implemented on a computer having a display device, e.g., a CRT (cathode ray tube) or LCD (liquid crystal display) monitor, for displaying information to the user and a keyboard and a pointing device, e.g., a mouse or a trackball, by which the user can provide input to the computer. Other kinds of devices can be used to provide for interaction with a user as well; for example, feedback provided to the user can be any form of sensory feedback, e.g., visual feedback, auditory feedback, or tactile feedback; and input from the user can be received in any form, including acoustic, speech, or tactile input. In addition, a computer can interact with a user by sending documents to and receiving documents from a device that is used by the user; for example, by sending web pages to a web browser on a user's client device in response to requests received from the web browser.

Embodiments of the subject matter described in this specification can be implemented in a computing system that includes a back end component, e.g., as a data server, or that includes a middleware component, e.g., an application server, or that includes a front end component, e.g., a client computer having a graphical user interface or a Web browser through which a user can interact with an implementation of the subject matter described in this specification, or any combination of one or more such back end, middleware, or front end components. The components of the system can be interconnected by any form or medium of digital data communication, e.g., a communication network. Examples of communication networks include a local area network (“LAN”) and a wide area network (“WAN”), an inter-network (e.g., the Internet), and peer-to-peer networks (e.g., ad hoc peer-to-peer networks).

The computing system can include clients and servers. A client and server are generally remote from each other and typically interact through a communication network. The relationship of client and server arises by virtue of computer programs running on the respective computers and having a client-server relationship to each other. In some embodiments, a server transmits data (e.g., an HTML page) to a client device (e.g., for purposes of displaying data to and receiving user input from a user interacting with the client device). Data generated at the client device (e.g., a result of the user interaction) can be received from the client device at the server.

While this specification contains many specific implementation details, these should not be construed as limitations on the scope of any inventions or of what may be claimed, but rather as descriptions of features specific to particular embodiments of particular inventions. Certain features that are described in this specification in the context of separate embodiments can also be implemented in combination in a single embodiment. Conversely, various features that are described in the context of a single embodiment can also be implemented in multiple embodiments separately or in any suitable subcombination. Moreover, although features may be described above as acting in certain combinations and even initially claimed as such, one or more features from a claimed combination can in some cases be excised from the combination, and the claimed combination may be directed to a subcombination or variation of a subcombination.

Similarly, while operations are depicted in the drawings in a particular order, this should not be understood as requiring that such operations be performed in the particular order shown or in sequential order, or that all illustrated operations be performed, to achieve desirable results. In certain circumstances, multitasking and parallel processing may be advantageous. Moreover, the separation of various system components in the embodiments described above should not be understood as requiring such separation in all embodiments, and it should be understood that the described program components and systems can generally be integrated together in a single software product or packaged into multiple software products.

While the present invention has been described at some length and with some particularity with respect to the several described embodiments, it is not intended that it should be limited to any such particulars or embodiments or any particular embodiment, but it is to be construed with references to the appended claims so as to provide the broadest possible interpretation of such claims in view of the prior art and, therefore, to effectively encompass the intended scope of the invention. Furthermore, the foregoing describes the invention in terms of embodiments foreseen by the inventor for which an enabling description was available, notwithstanding that insubstantial modifications of the invention, not presently foreseen, may nonetheless represent equivalents thereto.

What is claimed is:

1. A computer-based method for evaluating risk for workers comprising:
receiving, at a processor, a first signal from a first wearable device indicative of vertical acceleration detected by the first wearable device over time;
identifying, in the first signal, an indication of weightlessness at the first wearable device;
upon detecting the indication of weightlessness, determining an amount of time associated with the indication of weightlessness; and
determining a risk metric associated with the indication of weightlessness based on the amount of time associated with the indication of weightlessness.

2. The computer-based method of claim 1 wherein the first signal comprises an output of an accelerometer at the first wearable device, and wherein the indication of weightlessness is a reading of an absolute value of vertical acceleration below a threshold level.

3. The computer-based method of claim 2 wherein the amount of time associated with the indication of weightlessness is an amount of time for which the accelerometer outputs a reading of vertical acceleration below the threshold level.

4. The computer-based method of claim 1 wherein the determining of the risk metric associated with the indication of weightlessness includes considering an indication of weightlessness only if the amount of time is greater than a threshold amount of time.

5. The computer-based method of claim 4 wherein the determination of the risk metric associated with the indication of weightlessness is based on the amount of time associated with the indication of weightlessness reduced by the threshold amount of time.

6. The computer-based method of claim 1 wherein the amount of time corresponds to a height from which free fall is experienced, and wherein the indication of weightlessness is considered only if it corresponds to a height of greater than a threshold height.

7. The computer-based method of claim 6 wherein the threshold height is one foot and the indication of weightlessness is considered only if longer than 0.25 seconds.

8. The computer-based method of claim 6 wherein the threshold height is two feet and the indication of weightlessness is considered only if longer than 0.35 seconds.

9. The computer-based method of claim 6 wherein the threshold height corresponds to a defined piece of equipment utilized by a user. The computer-based method of claim 9 wherein the threshold height corresponds to a truck bed or a specified step of a ladder.

11. The computer-based method of claim 6 wherein the threshold height is based on which user of a plurality of potential users are wearing the first wearable device.

12. The computer-based method of claim 1 wherein the output of the accelerometer corresponds to local gravity during normal use and the indication of weightlessness corresponds to an output of the accelerometer lower than local gravity.

13. The computer-based method of claim 1 further comprising identifying, in the first signal, a plurality of secondary indications of weightlessness, each of which has a corresponding associated amount of time.

14. The computer-based method of claim 13 further comprising determining a cumulative risk metric associated with the indication of weightlessness and the plurality of secondary indications of weightlessness, wherein the determining of the cumulative risk metric considers only indications of weightlessness longer than a threshold amount of time.

15. The computer-based method of claim 14 wherein the cumulative risk metric is based on a cumulative amount of time associated with all of the indication of weightlessness and the plurality of secondary indications of weightlessness, and wherein, for any indication longer than threshold amount of time, the corresponding amount of time is reduced by the threshold amount of time prior to summing the amounts of time to determine the cumulative amount of time.

16. The computer-based method of claim 14 wherein the determination of the cumulative risk metric is based only on the plurality of indications of weightlessness and the associated amounts of time.

17. The computer-based method of claim 14 further comprising triggering an alert when the cumulative risk metric associated with the indication of weightlessness is determined to represent a high risk of injury.

18. The computer-based method of claim 1 further comprising triggering an alert when the risk metric associated with the indication of weightlessness is determined to represent a high risk of injury.

19. The computer-based method of claim 1 wherein the determination of the risk is based only on the indication of weightlessness and the associated amount of time.

20. The computer-based method of claim 1 wherein the first wearable device is not calibrated after application to the user.

21. The computer-based method of claim 1, further comprising identifying a contextual characteristic associated with the worker either simultaneous with or immediately preceding or following the indication of weightlessness.

22. The computer-based method of claim 21, wherein the contextual characteristic is identified in the first signal.

23. The computer-based method of claim 22, wherein the contextual characteristic is a horizontal acceleration or deceleration prior to the indication of weightlessness identified in the first signal.

24. The computer-based method of claim 23 further comprising determining that the worker was decelerating in a vehicle prior to the indication of weightlessness based on the horizontal acceleration or deceleration identified.

25. The computer-based method of claim 24, an alert to a supervisor or a recommendation that a characteristic of the vehicle be modified is generated based on the contextual characteristic and the indication of weightlessness.

26. The computer-based method of claim 22, wherein the contextual characteristic is a change of gait or a failure to perform an expected movement, and wherein the contextual characteristic indicates worker injury.

27. The computer-based method of claim 21 further comprising receiving, at the processor, a second signal indicative of a physical characteristic of the first wearable device simultaneous with or immediately preceding or following the indication of weightlessness, and wherein the contextual characteristic is identified in the second signal.

28. The computer-based method of claim 27, wherein the second signal indicates location data so as to identify a location of the indication of weightlessness.

29. The computer-based method of claim 28 further comprising retrieving risk metrics and associated contextual characteristics from a plurality of workers, and identifying in the contextual characteristics at least one location consistently associated with high risk.

30. The computer-based method of claim 21, wherein a physical activity is associated with the indication of weightlessness based on the contextual characteristic.
